(19) United States
(12) Patent Application Publication
(10) Pub. No.: US 2021/0085255 A1
(43) Pub. Date: Mar. 25, 2021

(54) Compression And Mapping Of Physiological Signals For Health Condition Detection

(71) Applicant: BEIJING SHUNYUAN KAIHUA TECHNOLOGY LIMITED, Beijing (CN)

(72) Inventors: Yan Vule, Port Moody (CA); Yoav Aminov, Kfar-Saba (IL); Artem Galeev, Vancouver (CA); Andrew J. Rosenthal, San Francisco, CA (US); Kongqiao Wang, Hefei (CN)

(21) Appl. No.: 16/582,552

(22) Filed: Sep. 25, 2019

(51) Int. Cl. A61B 5/00; A61B 5/024

(52) U.S. Cl. CPC A61B 5/7232; A61B 5/02438; A61B 5/681; A61B 5/0006; A61B 5/0024

(57) ABSTRACT

Physiological signals produced at a wearable device are compressed and mapped to detect a health condition of a user of the wearable device. Physiological signal data indicating a physical quality of the user of the wearable device is produced based on noisy data recorded using sensors of the wearable device. The physiological signal data is compressed at the wearable device using a dictionary defined at a server device. The server device receives and decompresses the compressed physiological signal data to produce denoised physiological signal data indicating the physical quality of the user of the wearable device. A change in a physiological state of the user of the wearable device is determined using the denoised physiological signal data and historical physiological data of the user of the wearable device. The health condition is detected based on the change in the physiological state of the user of the wearable device.

### TECHNICAL FIELD

This disclosure relates to the compression and mapping of physiological signals for health condition detection and personal health mapping.

### BACKGROUND

Persons at risk for various diseases may wish to monitor certain vital, or biometric, data, for example, to identify disease risk factors and predict diagnoses. Components for performing tests typically performed by traditional electrocardiograph, pulse oximeter, and other sensor devices as may be used for these tasks are becoming increasingly convenient due to their inclusion in wearable objects, such as wristbands, watches, and rings. For example, such a wearable device may include sensors for recording physiological measurements, which over time may be monitored to identify or predict health conditions of a user of the wearable device.

### SUMMARY

Disclosed herein are, inter alia, implementations of systems and techniques for the compression and mapping of physiological signals for health condition detection and personal health mapping.

In one implementation, a method for using compressed physiological signal data to detect a health condition of a user of a wearable device is disclosed. The method includes producing, by a software application run on the wearable device, physiological signal data based on noisy data recorded using one or more sensors of the wearable device. The physiological signal data indicates a physical quality of the user of the wearable device. The software application compresses the physiological signal data using a dictionary defined by a server application run on a server device. The compressed physiological signal data is communicated from the wearable device to the server device. The server application decompresses the compressed physiological signal data to produce denoised physiological signal data indicating the physical quality of the user of the wearable device. The server application determines a change in a physiological state of the user of the wearable device based on a difference between the physical quality of the user of the wearable device indicated by the denoised physiological signal data and historical physiological data of the user of the wearable device. The server application detects the health condition of the user of the wearable device based on the change in the physiological state of the user of the wearable device.

In another implementation, a system for using compressed physiological signal data to detect a health condition of a user of a wearable device is disclosed. The system comprises a wearable device, a server device, and an intermediary device. The wearable device includes one or more sensors. A software application run on the wearable device produces physiological signal data based on noisy data recorded using the one or more sensors. The physiological signal data indicates a physical quality of the user of the wearable device. The software application uses a dictionary to compress the physiological signal data. A server application run on the server device stores the compressed physiological signal data in a database. The server application detects the health condition by retrieving the stored compressed physiological signal data from the database and by decompressing the retrieved compressed physiological signal data. The intermediary device receives the compressed physiological signal data from the wearable device and that transmits the compressed physiological signal data to the server device.

In yet another implementation, a method for using compressed physiological signal data to detect a health condition of a user of a wearable device is provided. The method includes compressing, at the wearable device, a normalized pulse segment indicating a physical quality of the user of the wearable device. The normalized pulse segment is produced based on measurements recorded using one or more sensors of the wearable device. The compressed normalized pulse segment is communicated from the wearable device to a server device. The compressed normalized pulse segment is decompressed at the server device to produce denoised data indicating the physical quality of the user of the wearable device. A health profile associated with the user of the wearable device is updated at the server device based on the physical quality of the user of the wearable device indicated by the denoised data. The health condition of the user of the wearable device is detected at the server device using the updated health profile associated with the user of the wearable device and using a map of health conditions represents data associated with health profiles of users of multiple wearable devices.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a block diagram showing an example of a system for health condition detection.

FIG. 2 is a block diagram showing an example of a wearable device of a system for health condition detection.

FIG. 3 is a block diagram showing examples of a server device of a system for health condition detection.

FIG. 4 is a block diagram showing an example of a pipeline for updating a health profile associated with the user of the wearable device using physiological signal data.

FIG. 5 is a block diagram showing an example of a pipeline for using an updated health profile associated with a user of a wearable device to detect a health condition of the user of the wearable device.

FIG. 6 is a block diagram showing an example of a pipeline for using an updated map of health conditions to update a dictionary used for compressing physiological signal data.

FIG. 7 is an illustration showing an example of a map of health conditions.

FIG. 8 is a flowchart showing an example of a technique for using compressed physiological signal data to detect a health condition of a user of a wearable device.

FIG. 9 is a flowchart showing an example of a technique for producing physiological signal data for a user of a wearable device.

FIG. 10 is a flowchart showing an example of a technique for using an updated health profile associated with a user of a wearable device to identify or predict a health condition of the user of the wearable device.

FIG. 11 is a flowchart showing an example of a technique for using an updated map of health conditions to update a dictionary used for compressing physiological signal data.

FIG. 12 is a block diagram showing an example of a computing device which may be used in a system for health condition detection.

### DETAILED DESCRIPTION

Wearable devices are increasingly used to monitor physiological information of users, for example, heart rates, blood oxygen levels, and more. Many wearable devices record physiological measurements in response to user input, such as the user tapping a button or other interface element of the wearable device to cause the measurement to occur. In some cases, a wearable device may be configured to automatically record physiological measurements without manual user intervention. Wearable devices which automatically record physiological measurements are more likely to accurately capture information representative of the actual health of the user, for example, because the amount of data they capture is much larger. However, the larger amount of data captured requires larger amounts of computing resources to produce and process.

A typical wearable device used to record physiological measurements of a user does not include computing capabilities sufficient to analyze those measurements and/or to store deep histories of those measurements, for example, by detecting health conditions which may be present in the user based on those measurements. In many cases, those wearable devices communicate the recorded physiological measurements to a secondary computing device or system to perform the processing and analysis. However, where such a wearable device is also configured to automatically record the physiological measurements on a frequent or continuous basis, the communication of the large amounts of data making up those measurements to the secondary computing device or system presents a significant burden in terms of network bandwidth and computing power. Furthermore, the large amount of data communicated to the secondary computing device needs to be stored for future use, thereby presenting a significant burden in terms of storage requirement.

Implementations of this disclosure address problems such as these using physiological signals produced at a wearable device, which are compressed and mapped to detect a health condition of a user of the wearable device. Physiological signal data indicating a physical quality of the user of the wearable device is produced based on noisy data recorded using sensors of the wearable device. The physiological signal data is compressed at the wearable device using a dictionary defined at a server device. The server device receives and decompresses the compressed physiological signal data to produce denoised physiological signal data indicating the physical quality of the user of the wearable device. A change in a physiological state of the user of the wearable device is determined using the denoised physiological signal data and historical physiological data of the user of the wearable device. The health condition is detected based on the change in the physiological state of the user of the wearable device.

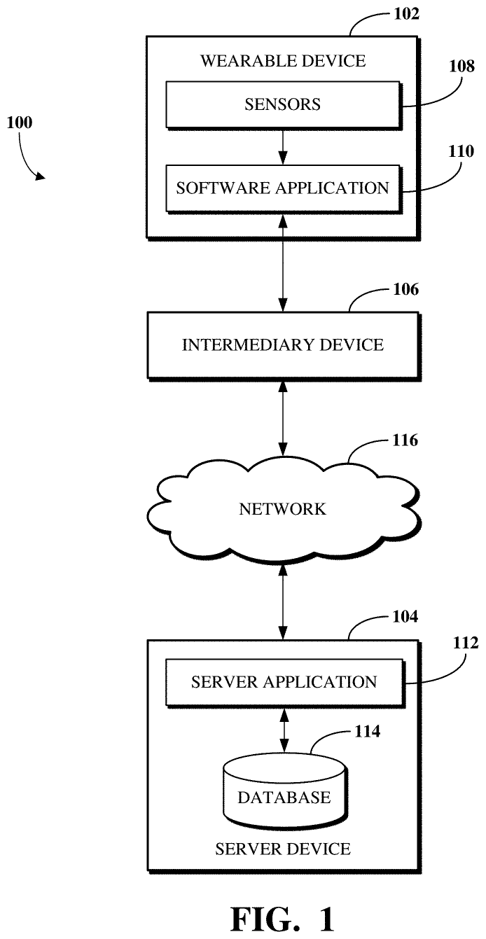

To describe some implementations in greater detail, reference is first made to examples of hardware and software structures used for the compressing and mapping physiological signals for health condition detection. FIG. 1 is a block diagram showing an example of a system 100 for health condition detection. The system 100 includes a wearable device 102, a server device 104, and an intermediary device 106 which is intermediate to the wearable device 102 and the server device 104.

The wearable device 102 is a computing device configured to be worn by a human user during operation. The wearable device 102 can be implemented as a bracelet, brace, wristband, arm band, leg band, ring, headband, necklace, or headset, or in another wearable device form factor. The wearable device 102 includes one or more sensors 108 used to record data indicative of physiological qualities of the user of the wearable device 102. The sensors 108 may include one or more of a photoplethysmogram (PPG) sensor, an electrocardiogram (ECG) sensor, an electrode, a pulse pressure sensor, a blood vessel property sensor, another sensor, or a combination thereof.

The wearable device 102 runs a software application 110 for processing physiological signal data produced based on the data recorded using the sensors 108. The software application 110 is application software used to produce physiological signal data based on the data recorded using the sensors 108 and to compress the physiological signal data, such as to prepare the physiological signal data for communication to the server device 104. The software application 110 can, for example, produce the physiological signal data by reconstructing a stream of pulses from measurements recorded using the sensors 108, performing pulse segmentation against the stream of pulses to identify a current pulse segment representing noisy data, and normalizing the current pulse segment to produce the physiological signal data. The software application 110 can compress the noisy signal using a dictionary defined at the server device 104. The software application 110 prepares the compressed physiological signal data for communication from the wearable device 102 to the server device 104.

The server device 104 is a computing device that runs a server application 112 for processing the compressed physiological signal data. The server device 104 may be or include a hardware server (e.g., a server device), a software server (e.g., a web server and/or a virtual server), or both. For example, where the server device 104 is or includes a hardware server, the server device 104 may be a server device located in a rack, such as of a data center.

The server application 112 is application software used to decompress the compressed physiological signal data produced at the wearable device 102 to detect a health condition of the user of the wearable device 102 using the decompressed physiological signal data. The server application 112 can, for example, receive the compressed physiological signal data from the intermediary device 106 and then decompress the compressed physiological signal data. The server application 112 can use the decompressed data to detect a health condition of the user of the wearable device 102. For example, the server application 112 can use the decompressed data to determine a change in a physiological state of the user and then detect the health condition of the user of the wearable device 102 based on the determined change.

The server application 112 accesses a database 114 on the server device 104 to perform at least some of the functionality of the server application 112. The database 114 is a database or other data store used to store, manage, or otherwise provide data used to deliver functionality of the server application 112. For example, the database 114 may store records or other data associated with one or more of health profiles associated with users of wearable devices (including a health profile associated with the user of the wearable device 102), the compressed physiological signal data received at the server device 104, information produced or otherwise determined by decompressing the compressed physiological signal data, dictionary entries used at the wearable device 102 to compress physiological signal data, update information used to update that dictionary, other data, or a combination thereof. The database 114 may, for example, be a relational database management system, an object database, an XML database, a configuration management database, a management information base, one or more flat files, other suitable non-transient storage mechanisms, or a combination thereof.

The intermediary device 106 is a device used to facilitate communications between the wearable device 102 and the server device 104. In particular, the intermediary device 106 receives data from the wearable device 102 and transmits that received data to the server device 104, such as for use by the server application 112. The intermediary device 106 may be a computing device, such as a mobile device (e.g., a smartphone, tablet, or other mobile device) or other computer. Alternatively, the intermediary device 106 may be or include network hardware, such as a router, a switch, a load balancer, another network device, or a combination thereof. As a further alternative, the intermediary device 106 may be another network-connected device. For example, the intermediary device 106 may be a network-connected power charger of the wearable device 102.

The intermediary device 106 may, for example, depending on the particular implementation thereof, run a companion application. The companion application is application software which configures the intermediary device 106 to transmit data to or receive data from the wearable device 102 and/or to transmit data to or receive data from the server device 104. The companion application can receive commands from a user of the intermediary device 106 (which typically is the same person as the user of the wearable device 102, but in some cases may not be the same person as the user of the wearable device 102). The companion application can receive commands from the user thereof through a user interface of the companion application. For example, where the intermediary device 106 is a computing device with a touch screen display, the command can be received by the user of the intermediary device 106 touching a portion of the display corresponding to a user interface element within the companion application.

For example, a command received by the companion application from the user of the intermediary device 106 may be a command to communicate compressed physiological signal data received at the intermediary device 106 (e.g., from the wearable device 102) to the server device 104. The intermediary device 106 transmits the compressed physiological signal data to the server device 104 responsive to such command. In another example, a command received by the companion application from the user of the intermediary device 106 may be a command to review information received from the server device 104, for example, relating to a health condition detected for the user of the wearable device 102.

The intermediary device 106 receives data from the wearable device 102 using a short-range communication protocol. For example, the short-range communication protocol may be Bluetooth®, Bluetooth® low energy, infrared, Z-Wave, ZigBee, another protocol, or a combination thereof. The intermediary device 106 transmits data received from the wearable device 102 to the server device 104 over a network 116. The network 116 may, for example, be a local area network, a wide area network, a machine-to-machine network, a virtual private network, or another public or private network. The network 116 may use a long-range communication protocol. For example, the long-range communication protocol may be Ethernet, TCP, IP, power line communication, Wi-Fi, GPRS, GSM, CDMA, another protocol, or a combination thereof.

The system 100 is used to continuously stream physiological signal data from the wearable device 102 to the server device 104. The sensors 108 may continuously, or otherwise on a frequent periodic basis, record measurements of physical qualities of the user of the wearable device 102. The compression of the physiological signal data produced for the recorded measurements allows for a reduction in bandwidth required to communicate the physiological signal data to the server device 104 and for a resource-efficient storage of the portions of the physiological signal data used to indicate the physical qualities within the database 114. The server application 112 retrieves and decompresses the compressed physiological signal data from within the database 114 to detect health conditions of the user of the wearable device 102 and to update records associated with the user of the wearable device 102. In this way, the system 100 may be used to continuously, or otherwise on a frequent periodic basis, monitor and update information relating to a physiological state of the user of the wearable device 102.

Implementations of the system 100 may differ from what is shown and described with respect to FIG. 1. In some implementations, the intermediary device 106 may be omitted. For example, the wearable device 102 may be configured to communicate directly with the server device 104 over the network 116. For example, the direct communication between the wearable device 102 and the server device 104 over the network 116 may include using a long range, low power system or another communication mechanism. In some implementations, the intermediary device 106 and the server device 104 may both be omitted. For example, the wearable device 102 may be configured to perform the functionality as described above with respect to the server device 104. In such an implementation, for example, the wearable device 102 may perform some or all of the compression and/or mapping functionality described above with respect to the server device 104. In such an implementation, the wearable device 102 may process and store data independent of other computing devices.

In some implementations, a client device may be given access to the server application 112. For example, the client device be a mobile device, such as a smart phone, tablet, laptop, or the like. In another example, the client device may be a desktop computer or another non-mobile computer. The client device may run client-side application software to communicate with the server application 112. For example, the client-side application software may be a mobile application that enables access to some or all functionality and/or data of the server application 112. The client device may, for example, communicate with the server device 104 over the network 116. In some such implementations, the client device may be the intermediary device 106.

In some implementations, the server device 104 may be a virtual server. For example, the virtual server can be implemented using a virtual machine (e.g., a Java virtual machine). The implementation of the virtual machine may use one or more virtual software systems, for example, an HTTP server, a Java servlet container, a hypervisor, or other software systems. In some such implementations, one or more of the virtual software systems used to implement the virtual server may instead be implemented in hardware.

In some implementations, the database 114 may be, include, or otherwise refer to multiple databases. For example, a first database can be a physiology database that stores compressed physiological signal data received at the server device 104, data associated with health profiles of users of wearable devices, and data used for detecting health conditions of those users (e.g., a map of health conditions, as will be described later). In another example, a second database can store information used by the dictionary at the wearable devices, such as dictionary entry information and update information used to update the dictionaries.

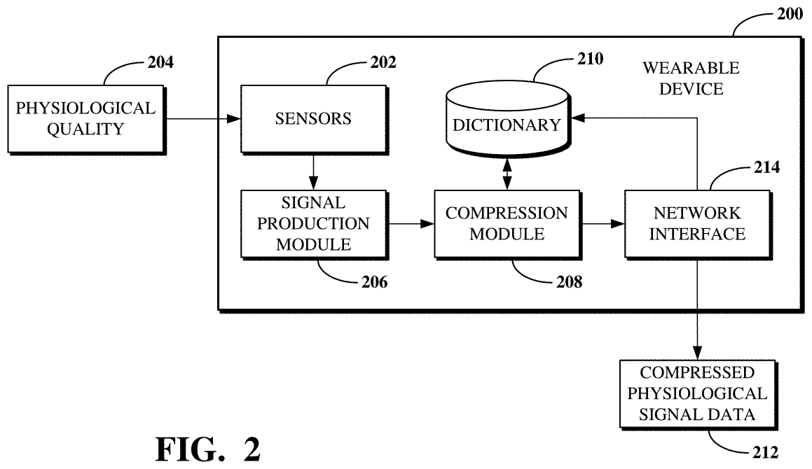

FIG. 2 is a block diagram showing an example of a wearable device 200 of a system for health condition detection. For example, the wearable device 200 may be the wearable device 102 of the system 100 shown in FIG. 1. The wearable device 200 includes sensors 202 which record and measure a physiological quality 204 received as input to the wearable device 200. The sensors 202 may, for example, be or include the sensors 108 shown in FIG. 1.

The physiological quality 204 is or refers to one or more physiological qualities of a user of the wearable device 200. The physiological quality 204 represents a measurable quality related to one or more vital systems of the body of the user of the wearable device 200, for example, the cardiovascular system, the respiratory system, the autonomic nervous system, or another system. For example, the physiological quality 204 may be or refer to one or more of a heart rate, a heart rate variability, a blood oxygen level, a blood pressure, or another physiological quality of the user of the wearable device 200.

A signal production module 206 represents functionality of a software application run on the wearable device 200 (e.g., the software application 110 shown in FIG. 1). The signal production module 206 uses measurements of the physiological quality 204 recorded using the sensors 202 to produce physiological signal data. The signal production module 206 has the ability to reconstruct the measurements of the physiological quality 204 recorded using the sensors 202 to produce a stream of pulses. For example, the signal production module 206 may reconstruct such measurements where distortion of the recorded data is introduced based on the physiological quality 204. Each of the pulses corresponds to a different time at which portions of the measurements of the physiological quality 204 are recorded using the sensors 202. The signal production module 206 performs pulse segmentation directly against the reconstructed stream. For example, the signal production module 206 can segment the stream of pulses to identify a current pulse segment of the stream of pulses. The signal production module 206 then normalizes the current pulse segment to produce a scaled one pulse raw or reconstructed signal including physiological signal data, which may include some measurement noise, and which represents a physiological state of the user of the wearable device 200 based on the physiological quality 204.

A compression module 208 represents other functionality of the software application run on the wearable device 200. The compression module 208 receives the physiological signal data produced using the signal production module 206. The physiological signal data received by the compression module 208 may, for example, be a string or array of data representing the physiological signal data in a one-dimensional format. The compression module 208 compresses the physiological signal data using a dictionary 210 to produce compressed physiological signal data 212. The compressed physiological signal data 212 may, for example, be a vector or other structure including N (e.g., three or four) numbers used to indicate the physical quality 204 in a latent space. Alternatively, the N numbers may indicate the physical quality as a two-dimensional or three-dimensional index in the dictionary 210. The dictionary 210 includes a number of dictionary entries and is defined by a server application run on a server device (e.g., the server application 112 shown in FIG. 1). For example, the compression module 208 can perform a dictionary look-up against the dictionary 210 based on the contents of the physiological signal data to identify a dictionary entry which corresponds to those contents. A dictionary entry may, for example, be or refer to a lookup table which incorporates standard information compression techniques. The dictionary entries may be defined or calculated online (e.g., using a connection to a server device, such as the server device 104 shown in FIG. 1) or offline.

Where a dictionary entry corresponding to the contents of the noisy signal are identified by the dictionary look-up, the compression module 208 compresses the contents of the physiological signal data based on that dictionary entry. Where a dictionary entry is not found, the compression module 208 performs pathological classification to determine how to compress the physiological signal data. For example, the pathological classification can include comparing individual pulse shapes within the physiological signal data to pulse shapes associated with dictionary entries. In another example, the pathological classification can include detecting sequence and/or rhythmic abnormalities in the physiological signal data, such as based on a learned model for the detection. The compression module 208 then uses the output of the pathological classification to compress the noisy signal. In some cases, the compression module 208 may include a flag in the compressed physiological signal data 212, such as to indicate that pathological classification was used for the compression thereof.

The classification and/or compression of the physiological signal data may use a neural network encoder or other machine learning approach for encoding the physiological signal data. As described above, the compressed physiological signal data 212 includes information representing the physical quality 204 in a latent space. Physiological information entering the latent space (e.g., by compression at the wearable device 200) can be used to uniquely identify the user of the wearable device 200. For example, the information representing the physical quality 204 in the latent space corresponds directly to the user of the wearable device 200 such that the information representing the physical quality 204 in the latent space is itself indicative of the identity of the user of the wearable device 200.

The network interface 214 is used to transmit information to and/or receive information from one or more devices external to the wearable device 200. The network interface 214 provides a connection or link to a network (e.g., the network 116 shown in FIG. 1). The network interface 210 is a wired or wireless network interface which may use different network protocols to connect with a device external to the wearable device 200. The wearable device 200 can use the network interface 214 to communicate with other devices over a short-range communication protocol or a long-range communication protocol.

For example, the network interface 214 can transmit the compressed physiological signal data 212 to an intermediary device (e.g., the intermediary device 106 shown in FIG. 1) over a short-range communication protocol. In another example, the network interface 214 can receive update information used to update the dictionary 210 from a server device (e.g., the server device 104 shown in FIG. 1) over a long-range communication protocol.

Implementations of the wearable device 200 may differ from what is shown and described with respect to FIG. 2. In some implementations, the signal production module 206 and the compression module 208 may be combined into a single software module. For example, the software application run on the wearable device 200 may include a single software module for both producing the physiological signal data based on measurements recorded using the sensors 204 and for compressing the physiological signal data using the dictionary 210. Alternatively, in some implementations, the functionality of one or both of the signal production module 206 or the compression module 208 may be distributed across more than one software module of the software application run on the wearable device 200.

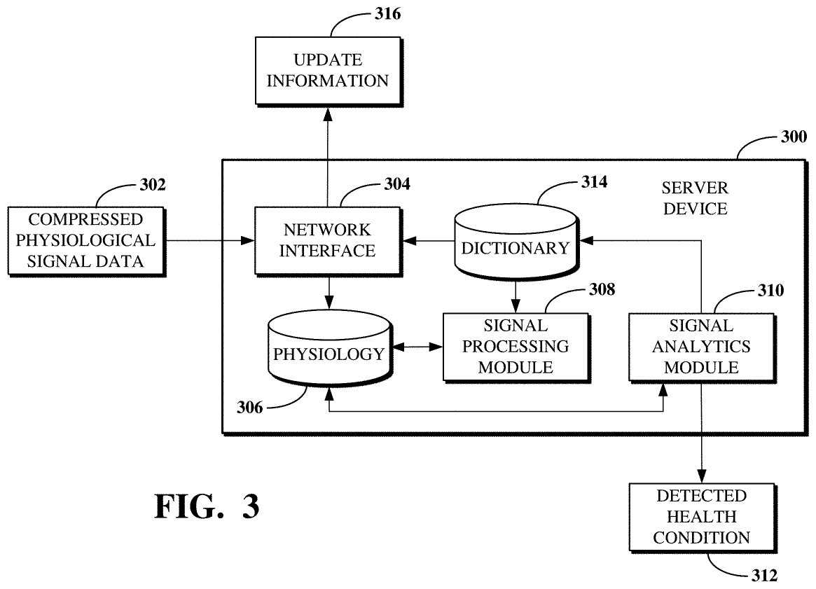

FIG. 3 is a block diagram showing examples of a server device 300 of a system for health condition detection. For example, the server device 300 may be the server device 104 of the system 100 shown in FIG. 1. The server device 300 receives compressed physiological signal data 302 using a network interface 304. For example, the compressed physiological signal data 302 can be the compressed physical signal data 212 shown in FIG. 2.

The network interface 304 is used to transmit information to and/or receive information from one or more devices external to the server device 300. The network interface 304 provides a connection or link to a network (e.g., the network 116 shown in FIG. 1). The network interface 300 is a wired or wireless network interface which may use different network protocols to connect with a device external to the server device 300. The server device 300 can use the network interface 304 to communicate with other devices over a short-range communication protocol or a long-range communication protocol.

For example, the network interface 304 can receive the compressed physiological signal data 302 from an intermediary device (e.g., the intermediary device 106 shown in FIG. 1) over a long-range communication protocol. In another example, as will be described later, the network interface 304 can transmit information for updating a used by the wearable device to compress physiological signal data produced thereat (e.g., the dictionary 210 shown in FIG. 2).

The server device 300 stores the compressed physiological signal data 302 in a physiology database 304. The physiology database 304 is a database (e.g., the database 114, or a portion thereof) which stores associated with health profiles of users of wearable devices, and data used for detecting health conditions of those users (e.g., a map of health conditions, as will be described later). The physiology database 304 is used to preserve information about physiological qualities of a user of the wearable device in a compressed form, for example, for later processing and/or analysis. Storage in the compressed form may improve resource usage for the processing and analysis of the compressed information.

A signal processing module 308 represents functionality of a server application run on the server device 300 (e.g., the server application 112 shown in FIG. 1). The signal processing module 308 retrieves the compressed physiological signal data 302 from within the physiology database 306. The signal processing module 308 then decompresses the retrieved compressed physiological signal data 302 to produce decompressed data, such as by using information stored within the dictionary 314. For example, the signal processing module 308 can use a neural network decoder or other machine learning approach for decoding the decompressed data from the compressed physiological signal data 302. The decompressed data, which includes the relevant information from the compressed physiological signal data 302 used to indicate the measured physiological quality of the user of the wearable device, can be used to uniquely identify the user of the wearable device. For example, the decompressed data corresponds directly to the user of the wearable device such that the decompressed data is itself indicative of the identity of the user of the wearable device. In another example, because the information stored within the dictionary 314 is based directly on the compressed physiological signal data 302, the dictionary entries within the dictionary 314 also correspond directly to the user of the wearable device such that some or all of the dictionary entries themselves are indicative of the identity of the user of the wearable device.

Whereas the compressed physiological signal data 302 includes noise, the decompressed data is a denoised form of the physiological signal data produced at the wearable device. The noise included in the compressed physiological signal data 302 at the wearable device may be removed from the compressed physiological signal data by the decompression thereof. As such, reconstructing the compressed physiological signal data 302 in some cases include discarding or otherwise removing portions of the original physiological signal data (e.g., portions which contain noise). The discarded portions are typically unperceivable by a user or during analysis at the signal analytics module 310.

A signal analytics module 310 represents other functionality of the server application run on the server device 300. The signal analytics module 310 uses the decompressed data output by the signal processing module 308 to detect a health condition 312 of the user of the wearable device. The signal analytics module 310 processes the decompressed data to determine a change in a physiological state of the user of the wearable device. In particular, the signal analytics module 310 compares historical physiological data of the user of the wearable device against information indicative of the physical quality measured at the wearable device within the decompressed data to determine the change in the physiological state of the user of the wearable device. The signal analytics module 310 then detects the health condition 312 of the user of the wearable device by mapping the change in the physiological state of the user of the wearable device to a map of health conditions.

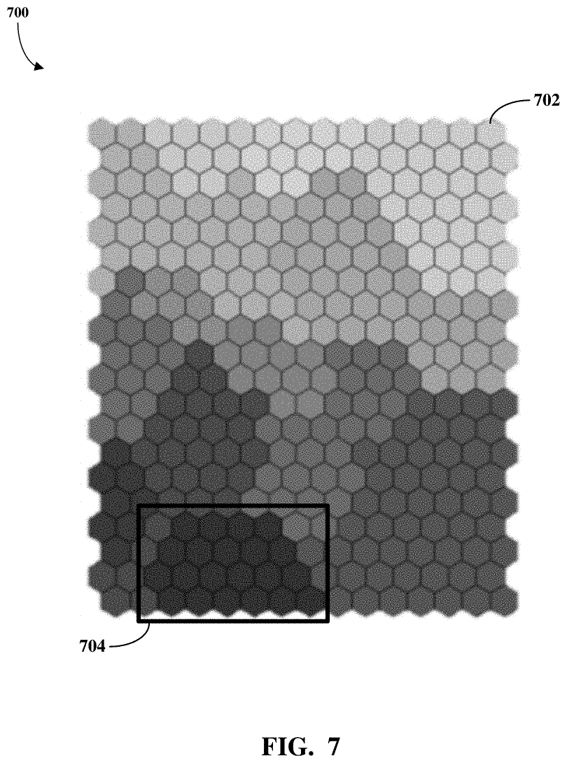

The map of health conditions is, includes, or otherwise refers to an organized set of information for classifying information about a physiology of a user of a wearable device (e.g., measurements of the physical quality of the user) as being demonstrative of one or more health conditions. For example, the map of health conditions can indicate that changes in a heart rate or changes in heart rate variability of a user of a wearable device over time may be demonstrative of a diagnosis or likelihood of developing ischemic heart disease. In another example, the map of health conditions can indicate that changes in blood oxygen level of the user of the wearable device over time may be demonstrative of a diagnosis or likelihood of developing a respiratory condition. Implementations and examples of a map of health conditions, including the generation and use of the map of health conditions, are described below with respect to FIG. 7.

The signal analytics module 310 further uses the compressed data output by the signal processing module 308 to update a health profile associated with the user of the wearable device. The health profile associated with the user of the wearable device is a collection of information related to physiological qualities measured for the user at the wearable device over time. The health profile may be stored within the physiology database 306. The health profile for each user is specific to the user and built over time based on further compressed physiological signal data received at the server device 300. Updating the health profile includes using the change in the physiological state of the user of the wearable device determined based on the decompressed data. For example, data associated with the physiological state of the user within the health profile can be identified and updated according to the determined change.

The signal analytics module 310 uses the decompressed data output by the signal processing module 308 or the health profile associated with the user of the wearable device, which is updated using the decompressed data output by the signal processing module 308, to update a dictionary used at the wearable device to compress physiological signal data. The dictionary used at the wearable device is defined based on entries within a dictionary database 314. The entries in the dictionary database 314 correspond to classifications of health conditions, for example, by indicating pulse segment templates which can be classified as relating to the health conditions.

The entries in the dictionary database 314 are determined based on information about health conditions as indicated in the map of health conditions. For example, the values that control the specific parameters for classifying a health condition may be defined based on the map of health conditions. When a change to the map of health conditions is made (e.g., based on updated health profiles of users, such as to refine classifications of the map of health conditions), information about the change can be used to determine whether an entry in the dictionary database 314 needs to be updated. In the event an entry needs to be updated, for example, because the classification for the entry does not match the new classification indicated by the updated map of health conditions, update information 316 indicating how to update that entry or otherwise indicating a replacement entry therefor is generated. The update information 316 can be transmitted from the server device 300 to a wearable device to cause an update to the dictionary used thereat.

Implementations of the server device 300 may differ from what is shown and described with respect to FIG. 3. In some implementations, the physiology database 306 and the dictionary database 314 may be combined into a single database. For example, the combined database may include separate tables or other structures used to separately maintain the compressed physiological signal data 302 received from an intermediary device (or from a wearable device, as the case may be) and the update information 316 to be transmitted to a wearable device.

In some implementations, the signal processing module 308 and the signal analytics module 310 may be combined into a single software module. For example, the server application run on the server device 300 may include a single software module for decompressing the compressed physiological signal data 302 and for detecting a health condition of a user of the wearable device from which the compressed physiological signal data 302 is received. Alternatively, in some implementations, the functionality of one or both of the signal processing module 308 or the signal analytics module 310 may be distributed across more than one software module of the server application run on the server device 300.

In some implementations, the signal analytics module 310 receives the output of the signal processing module 308 directly from the signal processing module 308. For example, instead of the signal processing module 308 storing the output thereof in the physiology database 306 and the signal analytics module 310 retrieving that stored output from the physiology database 306, the signal processing module 308 can transmit the output thereof to the signal analytics module 310. In another example, the signal analytics module 310 can pull the output of the signal processing module 308 therefrom without accessing the physiology database 306.

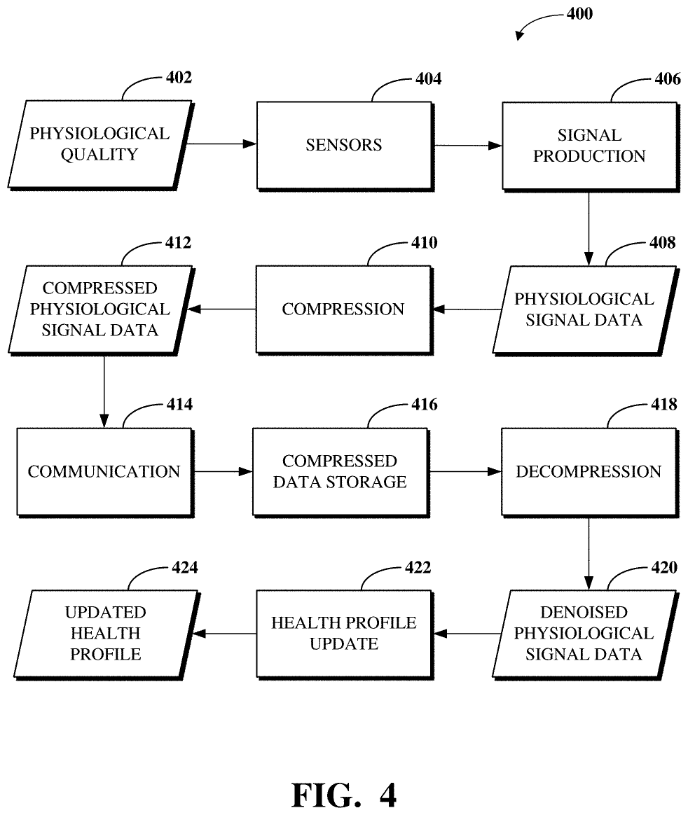

FIG. 4 is a block diagram showing an example of a pipeline 400 for updating a health profile associated with the user of the wearable device using physiological signal data. The pipeline 400 begins with the measuring of a physiological quality 402 of a user of a wearable device (e.g., the wearable device 200 shown in FIG. 2) using sensors 404 of the wearable device (e.g., the sensors 202). The measurements recorded using the sensors 404 are processed using signal production functionality 406 (e.g., by the signal production module 206) to produce physiological signal data 408. The physiological signal data 408 is compressed (e.g., by the compression module 208) to produce compressed physiological signal data 412 (e.g., the compressed physiological signal data 212).

The compressed physiological signal data 412 is communicated 414 from the wearable device to a server device, for example, using an intermediary device (e.g., the intermediary device 106 shown in FIG. 1). The compressed physiological signal data is stored 416 within a database at or otherwise accessible by the server device (e.g., the physiology database 306 shown in FIG. 3).

At a later time, which may be immediately after the storage of the compressed physiological signal data within the database or another time altogether (e.g., minutes, hours, days, weeks, or months after such storage), decompression 418 of the compressed physiological signal data occurs (e.g., by the signal processing module 308) to produce denoised physiological signal data 420. The denoised physiological signal data 420 is then used (e.g., by the signal analytics module 310) to update 422 a health profile associated with the user of the wearable device. The updated health profile 424 includes or otherwise indicates historical data associated with the user of the wearable device, for example, historical data indicative of a physiological state of the user of the wearable device, including data represented by the denoised physiological signal data 420.

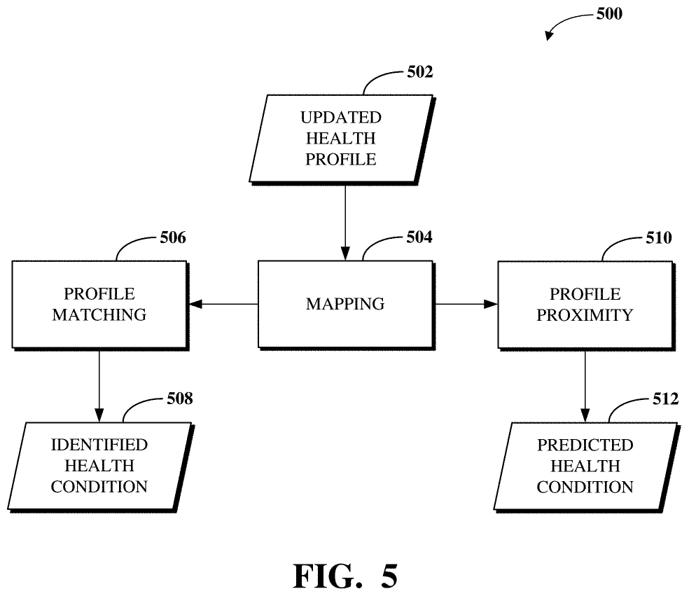

FIG. 5 is a block diagram showing an example of a pipeline 500 for using an updated health profile 502 associated with a user of a wearable device to detect a health condition of the user of the wearable device. The updated health profile 502 (e.g., the updated health profile 424 shown in FIG. 4) is processed using mapping functionality 504 (e.g., by the signal analytics module 310 shown in FIG. 3). Mapping the updated health profile 502 includes using a map of health conditions to identify or predict a health condition of the user of the wearable device. The pipeline 500 goes in one of two directions based on the application of the mapping functionality 504. Where profile matching 506 is performed, a health condition 508 is identified, for example, by matching the updated health profile 502 with a health profile of another user of another wearable device based on the map of health conditions. Where profile proximity 510 is performed, a health condition 512 is predicted based on the proximity within the map of health conditions of the updated health profile 502 to the health profile of the other user.

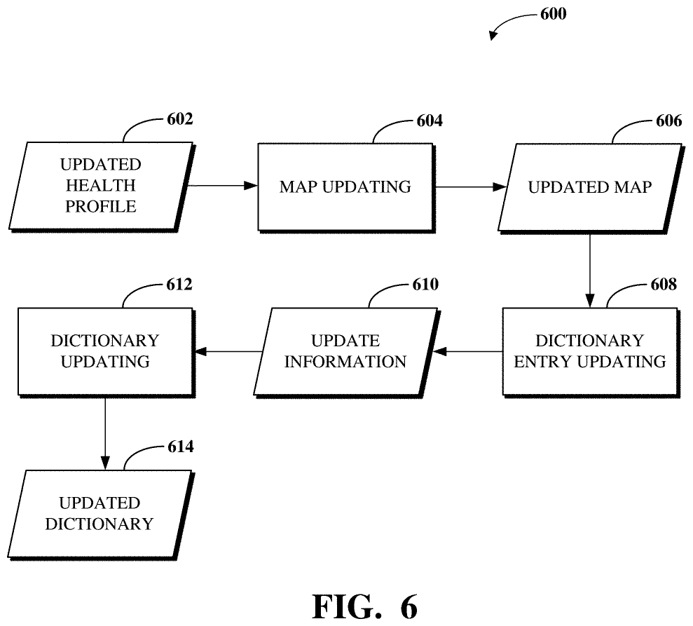

FIG. 6 is a block diagram showing an example of a pipeline 600 for using an updated map of health conditions to update a dictionary used for compressing physiological signal data. An updated health profile (e.g., the updated health profile 424 shown in FIG. 4) is used for map updating 604 (e.g., by the signal analytics module 310 shown in FIG. 3), such as to update the map of health conditions. The updated map of health conditions 606 is then used for dictionary entry updating 608 (e.g., by the signal analytics module 310), such as to determine how to update a dictionary used at a wearable device (e.g., the dictionary 210 and the wearable device 200 shown in FIG. 2) for compressing physiological signal data. Update information 610 (e.g., the update information 316) produced as a result of the dictionary entry updating 608 is then communicated from the server device (e.g., the server device 300) to the wearable device. The dictionary used at the wearable device is then updated 612 according to the update information to result in an updated dictionary 614.

FIG. 7 is an illustration showing an example of a map 700 of health conditions. As shown, the map 700 is visualized using a collection of hexagonal elements (e.g., the hexagonal element 702) in which adjacent hexagonal elements of a same color/shade correspond to a common health condition (e.g., the adjacent hexagonal elements 704). Other visualizations may be used to represent the map 700. Regardless of the visualization, the map 700 is, includes, or otherwise refers to a multi-dimensional structure used to organize information associated with health conditions detectable by a system which uses the map 700 (e.g., the system 100 shown in FIG. 1). The multi-dimensional structure may, for example, be a two-dimensional matrix of information in which the x-y position within the matrix corresponds to a particular health condition and the value at that x-y position indicates information about whether and/or the degree to which a user of a wearable device has the health condition.

Compressed physiological signal data received from a wearable device can indicate whether a health profile associated with a user maps to a particular position within the map 700. The compressed information represented in the latent space includes information used to indicate a physiological quality. The physical quality, by itself or when observed over time based on historical physiological data for a user of a wearable device, can indicate a health condition exists or is probable for the user. The particular mappings of physiological quality measurements to positions within the map 700 may be determined by modeling health conditions and physiological qualities related to those health conditions, for example, using a neural network or another machine learning approach.

Accordingly, a risk index for a user of a wearable device can be determined, identified, calculated, or otherwise obtained based on context from the map 700. A risk index is a high level calculation that aggregates all axes of the map 700 to a single meaningful number, which represents a medical risk of a user of a wearable device or information about such a medical risk. The risk index is determined, identified, calculated, or otherwise obtained based, for example, on a severity of one or more conditions indicated using the map 700, a total number of conditions indicated using the map 700, and/or other criteria. The risk may be determined, identified, calculated, or otherwise obtained and thereafter stored at a server which stores information about the map 700.

The map 700 is updated over time based on updates to health profiles of users of wearable devices. For example, classifications of health conditions indicated using the map 700 may be based on measurements of physiological qualities for the users of the wearable devices. The classifications can be extended to understanding how new sets of physiological signal data map to the map 700, for example, by identifying portions the map which match or are similar to the new sets of physiological signal data. The map 700 is created using existing data based on subspace (e.g., within the latent space), which is smaller bitwise but nonetheless includes the minimal information required for health condition monitoring. The mapping process may be performed using one or more encoding techniques, such as with relevant information for preserving feedback. The updating of the map 700 occurs based on demands for new information added to the database (e.g., information associated with a health condition) which is not well represented within the dictionary. The updating in such a case may, for example, include retraining and/or further encoding operations. Because the map 700 is created and updated based on information generated using sensors of a wearable device, the map 700 itself, as well as some or all of the information used to generate the map 700, is indicative of the identity of the user of the wearable device.

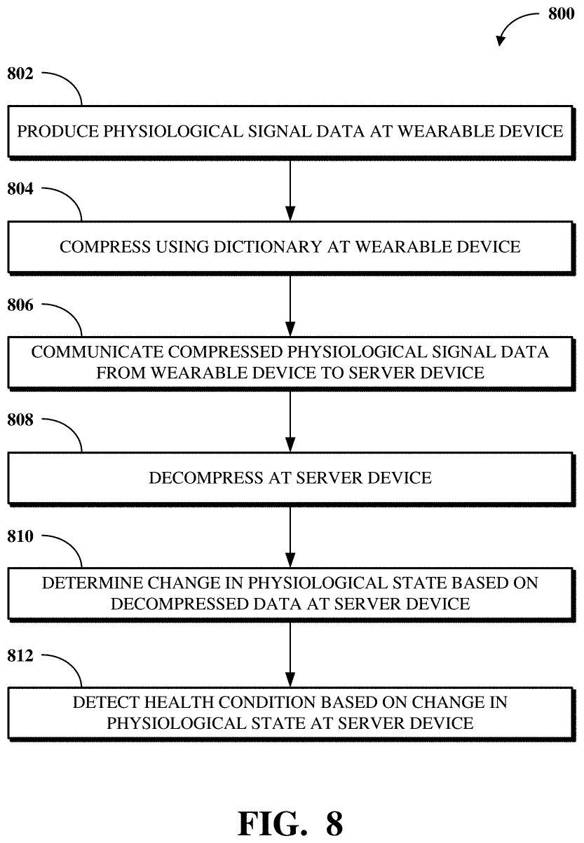

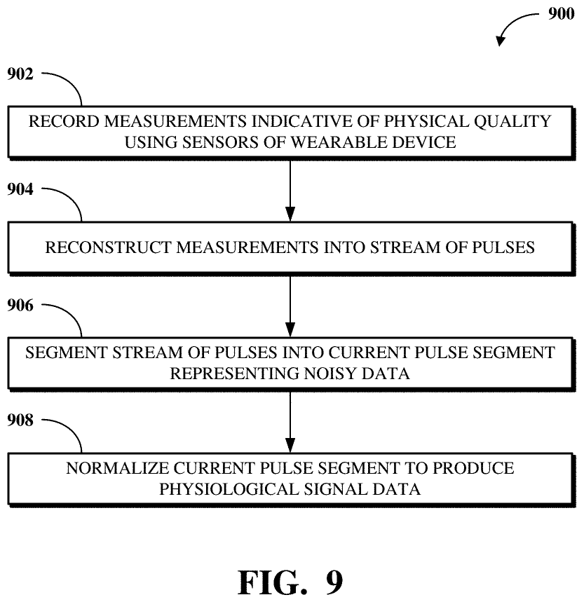

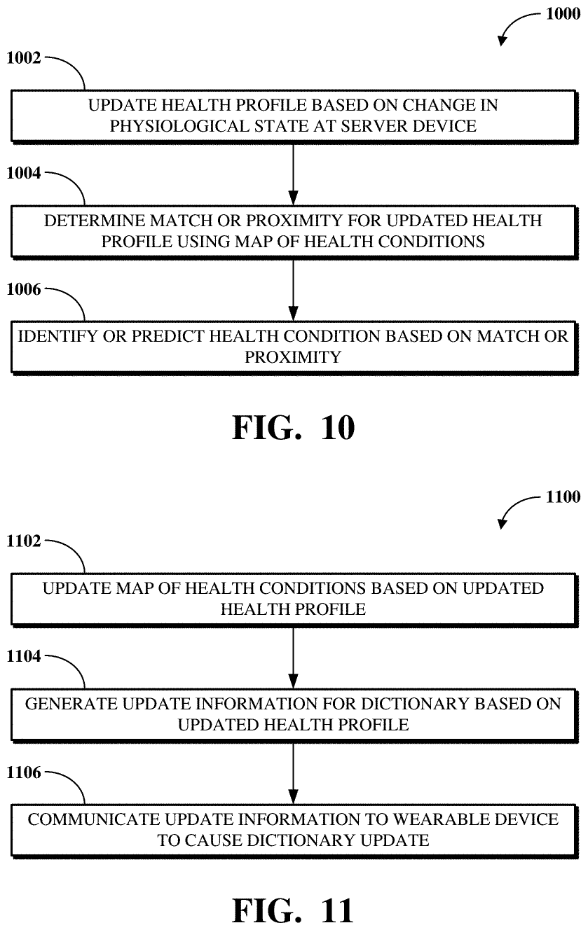

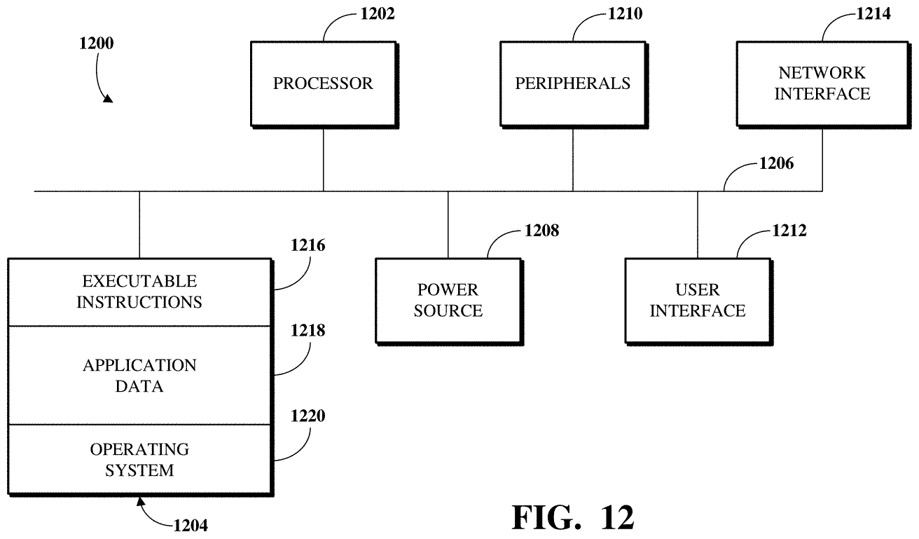

To further describe some implementations in greater detail, reference is next made to examples of techniques which may be performed for the compression and mapping of physiological signals for health condition detection. FIG. 8 is a flowchart showing an example of a technique 800 for using compressed physiological signal data to detect a health condition of a user of a wearable device. FIG. 9 is a flowchart showing an example of a technique 900 for producing physiological signal data for a user of a wearable device. FIG. 10 is a flowchart showing an example of a technique 1000 for using an updated health profile associated with a user of a wearable device to identify or predict a health condition of the user of the wearable device. FIG. 11 is a flowchart showing an example of a technique 1100 for using an updated map of health conditions to update a dictionary used for compressing physiological signal data.

The technique 800, the technique 900, the technique 1000, and/or the technique 1100 can be executed using computing devices, such as the systems, devices, hardware, and software described with respect to FIGS. 1-6. For example, each of the technique 800, the technique 900, the technique 1000, or the technique 1100 can be performed using one or more of the wearable device 102, the server device 104, or the intermediary device 106 of the system 100 shown in FIG. 1. The technique 800, the technique 900, the technique 1000, and/or the technique 1100 can be performed, for example, by executing a machine-readable program or other computer-executable instructions, such as routines, instructions, programs, or other code. The steps, or operations, of the technique 800, the technique 900, the technique 1000, the technique 1100, and/or another technique, method, process, or algorithm described in connection with the implementations disclosed herein can be implemented directly in hardware, firmware, software executed by hardware, circuitry, or a combination thereof.

For simplicity of explanation, the technique 800, the technique 900, the technique 1000, and the technique 1100, are each depicted and described herein as a series of steps or operations. However, the steps or operations in accordance with this disclosure can occur in various orders and/or concurrently. Additionally, other steps or operations not presented and described herein may be used. Furthermore, not all illustrated steps or operations may be required to implement a technique in accordance with the disclosed subject matter.

Referring first to FIG. 8, the technique 800 for using compressed physiological signal data to detect a health condition of a user of a wearable device is shown. At 802, physiological signal data is produced at a wearable device. The physiological signal data is data which indicates a physical quality of the user of the wearable device. The physiological signal data is produced by a software application run on the wearable device. The physiological signal data is produced based on noisy data recorded using one or more sensors of the wearable device. Implementations and examples of producing the physiological signal data for a user of a wearable device are described below with respect to FIG. 9.

At 804, the physiological signal data is compressed at the wearable device using a dictionary. The software application run on the wearable device uses the dictionary to compress the physiological signal data into compressed physiological signal data. Compressing the physiological signal data may include classifying the physiological signal data to determine a classification thereof and using a dictionary entry corresponding to the classification within the dictionary to compress the physiological signal data. For example, where the physiological signal data is or refers to a normalized pulse segment, the normalized pulse segment can be classified to determine a pulse classification for the normalized pulse segment. The physiological signal data can then be compressed using a dictionary entry which corresponds to the pulse classification within the dictionary. The dictionary is defined by a server application run on a server device and made available to the software application by the server application.

At 806, the compressed physiological signal data is communicated from the wearable device to the server device. For example, the compressed physiological signal data can be transmitted from the wearable device to the server device using an intermediary device intermediate to the wearable device and to the server device. For example, the compressed physiological signal data can be transmitted from the wearable device to an intermediary device using a short-range communication protocol. The compressed physiological signal data can then be transmitted from the intermediary device to the server device using a long-range communication protocol.

The communication of the compressed physiological signal data using the intermediary device may be completed without manual user intervention. For example, the intermediary device may be configured to automate the communication of the compressed physiological signal data to the server device, such as responsive to the receipt of the compressed physiological signal data at the intermediary device. Alternatively, the communication of the compressed physiological signal data using the intermediary device may be completed with manual user intervention. For example, a companion application run on the intermediary device may transmit the compressed physiological signal data to the server device responsive to receipt of a command from the user of the wearable device or the user of the intermediary device (where those users are different).

At 808, the compressed physiological signal data is decompressed at the server device. The server application run on the server device decompresses the compressed physiological signal data to produce decompressed data indicating the physical quality of the user of the wearable device. The decompressed data may be or refer to denoised physiological signal data. For example, the noise included in the noisy data used to produce the physiological signal data at the wearable device may be removed from the physiological signal data by the decompression thereof.

At 810, a change in a physiological state of the user of the wearable device is determined at the server device based on the decompressed data. The change in the physiological state of the user of the wearable device is determined based on a difference between the physical quality of the user of the wearable device indicated by the decompressed data and historical physiological data of the user of the wearable device. The historical physiological data of the user of the wearable device represents information about one or more physiological states of the user of the wearable device over time, such as based on multiple physiological signal data produced at different times at the wearable device. The historical physiological data of the user of the wearable is indicated in a health profile associated with the user of the wearable device. The health profile associated with the user of the wearable device is used to uniquely identify the user of the wearable device. Entries within the dictionary, which represent measurements in a latent space for the user of the wearable device, may also be used to uniquely identify the user of the wearable device. As such, determining the change in the physiological state of the user of the wearable device may include comparing the decompressed data against some or all of the historical physiological data indicated in the health profile associated with the user of the wearable device.

At 812, a health condition of the user of the wearable device is detected based on the change in the physiological state of the user of the wearable device. Detecting the health condition of the user of the wearable device can include comparing the change in the physiological state of the user of the wearable device or data indicative of such change against data which is associated with the health condition. For example, the server application can use information indicative of health conditions known to the server application to determine whether the user of the wearable device has one or more such health conditions based on similarities between the physiological state of the user of the wearable device, which has changed as indicated by the decompressed data, and the information indicative of the health conditions. Implementations and examples of detecting the health condition of the user of the wearable device are described below with respect to FIG. 10.

In some implementations, the technique 800 can include indicating the detection of the health condition to the user of the wearable device. For example, information indicative of the detection of the health condition can be made available to one or more of the software application run on the wearable device, a companion application run on the intermediary device, or the server application run on the server device. The one or more of the software application, the companion application, or the server application can output the information indicative of the detection of the health condition for display, such as at a display of the respective device.

In some implementations, communicating the compressed physiological signal data from the wearable device to the server device may omit the intermediary device. For example, the wearable device can directly transmit the compressed physiological signal data to the server device instead of using the intermediary device.

In some implementations, the technique 800 can include storing the compressed physiological signal data within a database responsive to the communication of the compressed physiological signal data to the server device. For example, the database may be a database hosted by the server device. In another example, the database may be a database not hosted by the server device, but which is nonetheless accessible by the server application run on the server device. The server application decompresses the compressed physiological signal data by first retrieving it from within the database.

In some implementations, the technique 800 can include using the detected health condition and/or the decompressed data to update the dictionary used at the wearable device to compress physiological signal data produced at the wearable device. Implementations and examples of updating the dictionary used for compressing physiological signal data are described below with respect to FIG. 11.

Referring next to FIG. 9, the technique 900 for producing physiological signal data for a user of a wearable device is shown. At 902, measurements indicative of a physical quality of a user of a wearable device are recorded using sensors of the wearable device. The measurements are or refer to data points usable to indicate the physical quality of the user of the wearable device. The measurements may include one or more measurements and may be recorded using one or more of the sensors of the wearable device.

At 904, the measurements are reconstructed into a stream of pulses. The stream of pulses is a data sequence which includes multiple pulses. Each pulse of the stream of pulses includes at least a portion of the measurements recorded using the sensors of the wearable device. Each pulse of the stream of pulses thus corresponds to a different time at which respective portions of the measurements were recorded using the sensors of the wearable device.

At 906, the stream of pulses is segmented into a current pulse segment representing noisy data. The stream of pulses is noisy in that it includes raw data recorded using the sensors of the wearable device. As such, the data represented by some or all pulses of the stream of pulses may include some noise. The stream of pulses is segmented into one or more segments to isolate portions of the measurements recorded using the sensors of the wearable device for further processing. One of the segments is referred to as a current pulse segment.

At 908, the current pulse segment is normalized to produce physiological signal data. The physiological signal data may then be compressed at the wearable device to prepare the physiological signal data for communication to a server device. Normalizing the current pulse segment can include fitting the current pulse segment with a universal set of parameters defined for processing measurements recorded using the sensors of the wearable device. The physiological signal data produced by normalizing the current pulse segment is thus produced according to parameters for further processing the physiological signal data.

Referring first to FIG. 10, the technique 1000 for using an updated health profile associated with a user of a wearable device to identify or predict a health condition of the user of the wearable device is shown. At 1002, a health profile associated with the user of the wearable device is updated. Updating the health profile associated with the user of the wearable device can include using a change in a physiological state of the user of the wearable device, or data indicative of such change.

For example, a physiological state of the user of the wearable device as previously indicated in the health profile associated with the user of the wearable device may be updated according to information included or otherwise indicated in decompressed data, which represents a decompressed form of physiological signal data produced at the wearable device. Where the health profile associated with the user of the wearable device includes or refers to multiple physiological states of the user of the wearable device, the physiological state associated with the physiological quality to which the decompressed data corresponds is identified and updated.

At 1004, a match or proximity for the updated health profile is determined using a map of health conditions. An example of a map of health conditions is described above with respect to FIG. 7. The map of health conditions represents data associated with health profiles of users of multiple wearable devices, including the user of the wearable device who is associated with the updated health profile. A health condition of the user of the wearable device can be identified or predicted using the map of health conditions and the updated health profile associated with the user of the wearable device.

Identifying the health condition of the user of the wearable device using the map of health conditions and the updated health profile associated with the user of the wearable device includes matching the updated health profile associated with the user of the wearable device to a health profile associated with another user of another wearable device. Alternatively, instead of matching the health profiles of the two users, relevant information (e.g., information relating to a same physiological state and/or physiological quality) included or otherwise indicated in those health profiles can be matched. The health profile associated with the other user of the other wearable device indicates that the user of the other wearable device has a particular health condition. The matching of the two health profiles, or of the data included or otherwise indicated in those two health profiles, can include determining that both health profiles, or the relevant data included or otherwise indicated in both health profiles, are represented at the same position of the map of health conditions.

Predicting the health condition of the user of the wearable device using the map of health conditions and the updated health profile associated with the user of the wearable device includes determining a proximity of the updated health profile associated with the user of the wearable device within the map of health conditions to a health profile associated with another user of another wearable device. Alternatively, instead of determining a proximity of the health profiles of the two users, a proximity of relevant information (e.g., information relating to a same physiological state and/or physiological quality) included or otherwise indicated in those health profiles can be determined. The health profile associated with the other user of the other wearable device indicates that the user of the other wearable device has a particular health condition. Determining the proximity of the two health profiles, or of the data included or otherwise indicated in those two health profiles, within the map of health conditions can include determining a distance between a position within the map of the updated health profile associated with the user of the wearable device (or of the relevant information thereof) and a position within the map of health conditions of the health profile associated with the other user of the other wearable device (or of the relevant information thereof).

At 1006, a health condition of the user of the wearable device is identified or predicted based on the match or proximity. For example, identifying the health condition can include determining that the user of the wearable device has the health condition based on the matching of the health profiles (or of relevant information thereof) of the user of the wearable device and another user of another wearable device who has the health condition. In another example, predicting the health condition can include determining a probability that the user of the wearable device has the health condition based on the proximity of the health profiles (or of relevant information thereof) within the map of health conditions of the user of the wearable device and of another user of another wearable device who has the health condition.

The probability that the user of the wearable device has the health condition can be a direct measure of the distance between the two health profiles (or of relevant information thereof) within the map of health conditions. For example, a difference in the positions of the relevant physiological quality measurements of those health profiles within the map of health conditions can be calculated. In another example, a lookup table may be used to identify the probability that the user of the wearable device has the health condition. For example, a lookup operation can be performed against the lookup table based on the distance between the two health profiles (or of relevant information thereof) within the map of health conditions to identify the probability corresponding to that distance within the lookup table.

Alternatively, the probability that the user of the wearable device has the health condition can be determined based on one or more thresholds. For example, such as where one threshold is used, if the distance between the two health profiles (or of relevant information thereof) within the map of health conditions meets a threshold, a determination can be made that the user of the wearable device has a high probability of having the health condition. In another example, such as where multiple thresholds are used, each of the thresholds can correspond to a different confidence level for the probability. For example, if the distance between the two health profiles (or of relevant information thereof) within the map of health conditions meets a first threshold but not a second threshold, a determination can be made that the user of the wearable device has a low probability of having the health condition; however, if the distance between the two health profiles (or of relevant information thereof) within the map of health conditions meets both of the first and second thresholds, a determination can be made that the user of the wearable device has a high probability of having the health condition.

Referring next to FIG. 11, the technique 1100 for using an updated map of health conditions to update a dictionary used for compressing physiological signal data is shown. At 1102, a map of health conditions is updated based on an updated health profile associated with a user of a wearable device. For example, information about the change in the physiological state of the user of the wearable device which was used to update the health profile of the user may be used to change a classification of a health condition within the map of health conditions. In another example, such change information as may be determined for multiple users of multiple wearable devices may be used to change a classification of a health condition within the map of health conditions.

At 1104, update information to use for updating a dictionary is generated based on the updated health profile. An entry corresponding to the portion of the map which was changed can be identified. For example, the entry within a dictionary database at the server device which corresponds to a classification updated within the map of health conditions can be identified. The entry can be changed according to the change made to the map of health conditions. The update information indicates the change made to the entry. Alternatively, the update information includes a replacement entry used to replace the identified entry.

At 1106, the update information is communicated to the wearable device to cause the dictionary used at the wearable device to update. The server device can communicate the update information directly to the wearable device to cause the update to the dictionary used at the wearable device. Updating the dictionary used at the wearable device using the update information can include identifying the entry within the dictionary at the wearable device which corresponds to the entry that has been changed within the dictionary database at the server device and then changing that entry within the dictionary at the wearable device according to the update information.

FIG. 12 is a block diagram showing an example of a computing device 1200 which may be used in a system for health condition detection, for example, the system 100 shown in FIG. 1. The computing device 1200 may be used to implement a server device on which a software application is run (e.g., the server device 104 and the server application 112 shown in FIG. 1). Alternatively, the computing device 1200 may be used to implement a wearable device that generates data which is processed using the software application run on the server device (e.g., the wearable device 102 shown in FIG. 1). As a further alternative, the computing device 1200 may be used to implement an intermediary device that communicates data between the wearable device and the server device (e.g., the intermediary device 106 shown in FIG. 1). As yet a further alternative, the computing device 1200 may be used as or to implement another device according to the implementations of this disclosure.

The computing device 1200 includes components or units, such as a processor 1202, a memory 1204, a bus 1206, a power source 1208, peripherals 1210, a user interface 1012, and a network interface 1214. One of more of the memory 1204, the power source 1208, the peripherals 1210, the user interface 1012, or the network interface 1214 can communicate with the processor 1202 via the bus 1206.

The processor 1202 is a central processing unit, such as a microprocessor, and can include single or multiple processors having single or multiple processing cores. Alternatively, the processor 1202 can include another type of device, or multiple devices, now existing or hereafter developed, configured for manipulating or processing information. For example, the processor 1202 can include multiple processors interconnected in some manner, including hardwired or networked, including wirelessly networked. For example, the operations of the processor 1202 can be distributed across multiple devices or units that can be coupled directly or across a local area or other suitable type of network. The processor 1202 can include a cache, or cache memory, for local storage of operating data or instructions.

The memory 1204 includes one or more memory components, which may each be volatile memory or non-volatile memory. For example, the volatile memory of the memory 1204 can be random access memory (RAM) (e.g., a DRAM module, such as DDR SDRAM) or another form of volatile memory. In another example, the non-volatile memory of the memory 1204 can be a disk drive, a solid state drive, flash memory, phase-change memory, or another form of non-volatile memory configured for persistent electronic information storage. The memory 1204 may also include other types of devices, now existing or hereafter developed, configured for storing data or instructions for processing by the processor 1202.

The memory 1204 can include data for immediate access by the processor 1202. For example, the memory 1204 can include executable instructions 1216, application data 1218, and an operating system 1220. The executable instructions 1216 can include one or more application programs, which can be loaded or copied, in whole or in part, from non-volatile memory to volatile memory to be executed by the processor 1202. For example, the executable instructions 1216 can include instructions for performing some or all of the techniques of this disclosure. The application data 1218 can include user data, database data (e.g., database catalogs or dictionaries), or the like. The operating system 1220 can be, for example, Microsoft Windows®, Mac OS X®, or Linux®, an operating system for a small device, such as a smartphone or tablet device; or an operating system for a large device, such as a mainframe computer.

The power source 1208 includes a source for providing power to the computing device 1200. For example, the power source 1208 can be an interface to an external power distribution system. In another example, the power source 1208 can be a battery, such as where the computing device 1200 is a mobile device or is otherwise configured to operate independently of an external power distribution system.

The peripherals 1210 includes one or more sensors, detectors, or other devices configured for monitoring the computing device 1200 or the environment around the computing device 1200. For example, the peripherals 1210 can include a geolocation component, such as a global positioning system location unit. In another example, the peripherals can include a temperature sensor for measuring temperatures of components of the computing device 1200, such as the processor 1202.

The user interface 1012 includes one or more input interfaces and/or output interfaces. An input interface may, for example, be a positional input device, such as a mouse, touchpad, touchscreen, or the like; a keyboard; or another suitable human or machine interface device. An output interface may, for example, be a display, such as a liquid crystal display, a cathode-ray tube, a light emitting diode display, or other suitable display.

The network interface 1214 provides a connection or link to a network (e.g., the network 116 shown in FIG. 1). The network interface 1214 can be a wired network interface or a wireless network interface. The computing device 1200 can communicate with other devices via the network interface 1214 using one or more network protocols, such as using Ethernet, TCP, IP, power line communication, Wi-Fi, Bluetooth, infrared, GPRS, GSM, CDMA, Z-Wave, ZigBee, another protocol, or a combination thereof.

Implementations of the computing device 1200 may differ from what is shown and described with respect to FIG. 12. In some implementations, the computing device 1200 can omit the peripherals 1210. In some implementations, the memory 1204 can be distributed across multiple devices. For example, the memory 1204 can include network-based memory or memory in multiple clients or servers performing the operations of those multiple devices. In some implementations, the application data 1218 can include functional programs, such as a web browser, a web server, a database server, another program, or a combination thereof. In some implementations, the computing device 1200 can omit the operating system 1220.

The implementations of this disclosure can be described in terms of functional block components and various processing operations. Such functional block components can be realized by a number of hardware or software components that perform the specified functions. For example, the disclosed implementations can employ various integrated circuit components (e.g., memory elements, processing elements, logic elements, look-up tables, and the like), which can carry out a variety of functions under the control of one or more microprocessors or other control devices. Similarly, where the elements of the disclosed implementations are implemented using software programming or software elements, the systems and techniques can be implemented with a programming or scripting language, such as C, C++, Java, JavaScript, assembler, or the like, with the various algorithms being implemented with a combination of data structures, objects, processes, routines, or other programming elements.

Functional aspects can be implemented in algorithms that execute on one or more processors. Furthermore, the implementations of the systems and techniques disclosed herein could employ a number of conventional techniques for electronics configuration, signal processing or control, data processing, and the like. The words “mechanism” and “component” are used broadly and are not limited to mechanical or physical implementations, but can include software routines in conjunction with processors, etc.

Likewise, the terms “system,” “module,” or “mechanism” as used herein and in the figures, and/or based on the context of such usage, may be understood as corresponding to a functional unit implemented using software, hardware (e.g., an integrated circuit, such as an ASIC), or a combination of software and hardware. In certain contexts, such systems, modules, or mechanisms may be understood to be a processor-implemented software system, processor-implemented software module, or processor-implemented software mechanism that is part of or callable by an executable program, which may itself be wholly or partly composed of such linked systems, modules, or mechanisms.

Implementations or portions of implementations of the above disclosure can take the form of a computer program product accessible from, for example, a computer-usable or computer-readable medium. A computer-usable or computer-readable medium can be a device that can, for example, tangibly contain, store, communicate, or transport a program or data structure for use by or in connection with a processor. The medium can be, for example, an electronic, magnetic, optical, electromagnetic, or semiconductor device.

Other suitable mediums are also available. Such computer-usable or computer-readable media can be referred to as non-transitory memory or media, and can include volatile memory or non-volatile memory that can change over time. A memory of an apparatus described herein, unless otherwise specified, does not have to be physically contained by the apparatus, but is one that can be accessed remotely by the apparatus, and does not have to be contiguous with other memory that might be physically contained by the apparatus.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method for using compressed physiological signal data to detect a health condition of a user of a wearable device, the method comprising:
producing, by a software application run on the wearable device, physiological signal data based on noisy data recorded using one or more sensors of the wearable device, the physiological signal data indicating a physical quality of the user of the wearable device;
compressing, by the software application, the physiological signal data using a dictionary defined by a server application run on a server device;
communicating, from the wearable device, the compressed physiological signal data to the server device;
decompressing, by the server application, the compressed physiological signal data to produce denoised physiological signal data indicating the physical quality of the user of the wearable device;
determining, by the server application, a change in a physiological state of the user of the wearable device based on a difference between the physical quality of the user of the wearable device indicated by the denoised physiological signal data and historical physiological data of the user of the wearable device; and
detecting, by the server application, the health condition of the user of the wearable device based on the change in the physiological state of the user of the wearable device.

2. The method of claim 1, wherein producing the physiological signal data based on the noisy data recorded using the one or more sensors of the wearable device comprises:
recording, using the one or more sensors of the wearable device, measurements indicative of the physical quality of the user of the wearable device;
reconstructing the measurements into a stream of pulses, wherein each pulse of the stream of pulses corresponds to a different time at which portions of the measurements are recorded;
segmenting the stream of pulses into one or more pulse segments including a current pulse segment representing the noisy data; and
normalizing the current pulse segment to produce the physiological signal data.

3. The method of claim 2, wherein compressing the physiological signal data using the dictionary defined by the server application run on the server device comprises:
classifying the normalized current pulse segment to determine a pulse classification for the normalized current pulse segment; and
compressing the physiological signal data using a dictionary entry corresponding to the pulse classification within the dictionary.

4. The method of claim 3, wherein the normalized current pulse segment is classified and compressed using a neural network encoder, wherein the compressed physiological signal data is decompressed using a neural network decoder, wherein the compressed physiological signal data represents the physical quality of the user of the wearable device in a latent space.

5. The method of claim 4, further comprising:
responsive to the server application receiving the compressed physiological signal data, storing, by the server application, data represented in the latent space and indicating the physical quality of the user within a database,
wherein decompressing the compressed physiological signal data to produce the denoised signal comprises:
retrieving, by the server application, the data represented in the latent space and indicating the physical quality of the user from the database.

6. The method of claim 1, wherein the historical physiological data of the user of the wearable device is indicated in a health profile associated with the user of the wearable device, wherein the health profile associated with the user of the wearable device is used to uniquely identify the user of the wearable device, the method further comprising:
updating, by the server application, the health profile associated with the user of the wearable device based on the change in the physiological state of the user of the wearable device.

7. The method of claim 6, wherein a map of health conditions represents data associated with health profiles of users of multiple wearable devices, wherein the health profiles of the users of the multiple wearable devices includes the health profile associated with the user of the wearable device.

8. The method of claim 7, wherein detecting the health condition of the user based on the change in the physiological state of the user of the wearable device comprises:
matching, using the map of health conditions, the updated health profile associated with the user of the wearable device to a health profile associated with another user of another wearable device, wherein the health profile associated with the other user of the other wearable device indicates that the user of the other wearable device has the health condition; and
determining that the user of the wearable device has the health condition based on the matching.

9. The method of claim 7, wherein detecting the health condition of the user based on the change in the physiological state of the user of the wearable device comprises:
determining a proximity of the updated health profile associated with the user of the wearable device within the map of health conditions to a health profile associated with another user of another wearable device, wherein the health profile associated with the other user of the other wearable device indicates that the user of the other wearable device has the health condition; and
determining a probability that the user of the wearable device has or will have the health condition based on the proximity.

10. The method of claim 7, further comprising:
updating, based on the updated health profile associated with the user of the wearable device, the map of health conditions;
generating update information for the dictionary based on the updated map of health conditions; and
communicating, from the server application, the update information to the wearable device to cause an update to the dictionary.

11. The method of claim 1, wherein communicating the compressed physiological signal data to the server device comprises:
transmitting, using a short-range communication protocol, the compressed physiological signal data from the wearable device to an intermediary device; and
transmitting, using a long-range communication protocol, the compressed physiological signal data from the intermediary device to the server device.

12. A system for using compressed physiological signal data to detect a health condition of a user of a wearable device, the system comprising:
a wearable device including one or more sensors, wherein a software application run on the wearable device produces physiological signal data based on noisy data recorded using the one or more sensors, wherein the physiological signal data indicates a physical quality of the user of the wearable device, wherein the software application uses a dictionary to compress the physiological signal data;
a server device, wherein a server application run on the server device stores the compressed physiological signal data in a database, wherein the server application detects the health condition by retrieving the stored compressed physiological signal data from the database and by decompressing the retrieved compressed physiological signal data; and
an intermediary device that receives the compressed physiological signal data from the wearable device and that transmits the compressed physiological signal data to the server device.

13. The system of claim 12, wherein the software application produces the physiological signal data based on the noisy data recorded using the one or more sensors by:
recording, using the one or more sensors, measurements indicative of the physical quality of the user of the wearable device;
reconstructing the measurements into a stream of pulses, wherein each pulse of the stream of pulses corresponds to a different time at which portions of the measurements are recorded;
segmenting the stream of pulses into one or more pulse segments including a current pulse segment representing the noisy data; and
normalizing the current pulse segment to produce the physiological signal data.

14. The system of claim 13, wherein the software application uses the dictionary to compress the physiological signal data by:
classifying the normalized current pulse segment to determine a pulse classification for the normalized current pulse segment; and
compressing the physiological signal data using a dictionary entry corresponding to the pulse classification within the dictionary.

15. The system of claim 12, wherein the server application detects the health condition by retrieving the stored compressed physiological signal data from the database and by decompressing the retrieved compressed physiological signal data by:
decompressing the retrieved compressed physiological signal data to produce denoised physiological signal data indicating the physical quality of the user of the wearable device;
determining a change in a physiological state of the user of the wearable device based on a difference between the physical quality of the user of the wearable device indicated by the denoised physiological signal data and a health profile associated with the user of the wearable device; and
detecting the health condition of the user based on the change in the physiological state of the user of the wearable device.

16. The system of claim 15, wherein the server application updates the health profile associated with the user of the wearable device based on the change in the physiological state of the user of the wearable device,
wherein the server application maintains a map of health conditions represents data associated with health profiles of users of multiple wearable devices, wherein the health profiles of the users of the multiple wearable devices includes the health profile associated with the user of the wearable device,
wherein the server application detects the health condition of the user based on the change in the physiological state of the user of the wearable device by using the updated health profile associated with the user of the wearable device and by using the map of health conditions.

17. A method for using compressed physiological signal data to detect a health condition of a user of a wearable device, the method comprising:
compressing, at the wearable device, a normalized pulse segment indicating a physical quality of the user of the wearable device, the normalized pulse segment produced based on measurements recorded using one or more sensors of the wearable device;
communicating the compressed normalized pulse segment from the wearable device to a server device;
decompressing, at the server device, the compressed normalized pulse segment to produce denoised data indicating the physical quality of the user of the wearable device;
updating, at the server device, a health profile associated with the user of the wearable device based on the physical quality of the user of the wearable device indicated by the denoised data; and
detecting, at the server device, the health condition of the user of the wearable device using the updated health profile associated with the user of the wearable device and using a map of health conditions represents data associated with health profiles of users of multiple wearable devices.

18. The method of claim 17, wherein detecting the health condition of the user using the updated health profile associated with the user of the wearable device and using a map of health conditions represents data associated with health profiles of users of multiple wearable devices comprises:
matching, using the map of health conditions, the updated health profile associated with the user of the wearable device to a health profile associated with another user of another wearable device, wherein the health profile associated with the other user of the other wearable device indicates that the user of the other wearable device has the health condition; and
determining that the user of the wearable device has the health condition based on the matching.

19. The method of claim 17, wherein detecting the health condition of the user using the updated health profile associated with the user of the wearable device and using a map of health conditions represents data associated with health profiles of users of multiple wearable devices comprises:
determining a proximity of the updated health profile associated with the user of the wearable device within the map of health conditions to a health profile associated with another user of another wearable device, wherein the health profile associated with the other user of the other wearable device indicates that the user of the other wearable device has the health condition; and
determining a probability that the user of the wearable device has or will have the health condition based on the proximity.

20. The method of claim 17, wherein the normalized pulse segment is compressed using a dictionary defined at the server device, wherein the dictionary is updated by the server device based on the updated health profile associated with the user of the wearable device.
